(19) United States
(12) Patent Application Publication
(10) Pub. No.: US 2018/0085026 A1
(43) Pub. Date: Mar. 29, 2018

(54) APPARATUS AND METHOD FOR MEASURING ELECTRICAL CHARACTERISTIC USING NUCLEAR MAGNETIC RESONANCE

(71) Applicant: Hitachi, Ltd., Tokyo (JP)

(72) Inventors: Yukio KANEKO, Tokyo (JP); Hisaaki OCHI, Tokyo (JP); Yoshihisa SOUTOME, Tokyo (JP)

(21) Appl. No.: 15/701,640

(22) Filed: Sep. 12, 2017

(30) Foreign Application Priority Data
Sep. 29, 2016 (JP) 2016-191281

(51) Int. Cl. A61B 5/053; A61B 5/055; A61B 5/00; G01R 33/24

(52) U.S. Cl. CPC A61B 5/0537; A61B 5/0536; G01R 33/24; A61B 5/7278; A61B 5/055

(57) ABSTRACT

When an electrical characteristic of a predetermined region of a subject placed in a static magnetic field space is measured by using magnetic resonance signals measured from the region, measurement data measured by coinciding direction of a tissue structure of the subject with the direction of the static magnetic field, and measurement data measured with a direction of the tissue structure of the subject crossing the direction of the static magnetic field are used. A rotating magnetic field map of the region is created from the measurement data, and the electrical characteristic is calculated by using the rotating magnetic field map. The electrical characteristic is calculated as an electrical characteristic including anisotropy by using information about the direction of tissue structure. According to the present invention, electrical characteristic such as electrical conductivity including anisotropy can be measured with good precision with an electrical characteristic measuring apparatus using nuclear magnetic resonance.

### INCORPORATION BY REFERENCE

The present application claims priority from Japanese patent application JP-2016-191281 filed on Sep. 29, 2016, the content of which is hereby incorporated by reference into this application.

### TECHNICAL FIELD

The present invention relates to an apparatus and method for measuring an electrical characteristic of human bodies or the like such as electrical conductivity and permittivity, especially a technique for measuring magnetic resonance signals, and calculating an electrical characteristic including anisotropy from the magnetic resonance signals.

### BACKGROUND ART

Electrical characteristics of human bodies such as electrical conductivity have conventionally been measured by flowing a weak electric current through human bodies. However, any electrical characteristic, i.e., electrical characteristic image, cannot be obtained for each tissue by such a method. Under such a technical situation, a method for measuring an electrical characteristic using a magnetic resonance imaging apparatus (MRI apparatus) has been developed in recent years (for example, Patent documents 1 and 2).

In the technique described in Patent document 1, measurement is performed with flowing or without flowing a weak radio frequency current with different phases of the radio frequency magnetic field for exciting spins constituting a tissue of a subject, and electrical conductivity and permittivity images are generated from the obtained four images by operations for every pixel. This method is invasive, since it requires electrification. It also suffers from a problem that flowing an electric current in a deep part of a living body is difficult. In the technique described in Patent document 2, on the other hand, distributions of permittivity and electrical conductivity are calculated by obtaining solutions of the Maxwell's equations using electrical field intensity distribution of the electromagnetic field applied for generating magnetic resonance signals, and intensity distribution of magnetic induction field induced thereby. With this technique, an electrical characteristic image can be obtained in a non-invasive manner. Further, Non-patent document 1 proposes a technique of measuring diffusion coefficient with an MRI apparatus, and presuming electrical conductivity from the diffusion coefficient.

### PRIOR ART REFERENCES

### Patent Documents

Patent document 1: Japanese Patent Unexamined Publication (KOKAI) No. 2009-119204
Patent document 2: Japanese Patent Unexamined Publication (KOHYO) No. 2009-504224

### Non-Patent Document

Non-patent document 1: Tuch D. S. et al., Conductivity tensor mapping of the human brain using diffusion tensor MRI, PNAS, 2001, vol. 98, No. 20, pp. 11697-11701

### SUMMARY OF THE INVENTION

### Object to be Achieved by the Invention

Electrical conductivity or permittivity of living body also correlates with structure of tissue, and is not necessarily isotropic. It is important to know an electrical characteristic including anisotropy for knowing details of structure of tissue or reactions of tissue to electromagnetic fields generated by various instruments and measurement apparatuses.

In the technique described in Non-patent document 1, electrical conductivity including anisotropy is calculated on the assumption that electrical conductivity correlates with diffusion coefficient. However, the electrical conductivity obtained by this technique is indirectly estimated electrical conductivity, and it is still indefinite whether it is reasonable to uniformly apply such an estimate equation to the whole body tissues of a subject as the object. The technique described in Patent document 2 is basically configured on assumption that electrical characteristic is isotropic. Although Patent document 2 briefly refers to anisotropy, it does not specifically propose any technique for measuring anisotropy with sufficient accuracy.

Therefore, an object of the present invention is to measure anisotropy of electrical characteristic such as electrical conductivity and permittivity with sufficient accuracy.

### Means for Achieving the Object

The present invention is based on a technique of calculating an electrical characteristic from a rotating magnetic field calculated from magnetic resonance signals, and is based on an discovery that, as for such an electrical characteristic as calculated in such a manner, components of a certain axis of the coordinate system of the magnetic resonance imaging apparatus are most accurately measured. Therefore, according to the present invention, relation between direction of tissue structure and a predetermined axis of the coordinate system is recorded, and an electrical characteristic including anisotropy is calculated on the basis of the relation. Alternatively, two or more sets of measurement data for different directions of tissue structure different from the direction of the predetermined axis of the coordinate system are obtained, and an electrical characteristic including anisotropy is calculated by calculation with these measurement data.

Thus, the electrical characteristic measuring apparatus of the present invention comprises a measurement part that measures a magnetic resonance signal emitted from a subject, a storage part that stores information concerning direction of tissue structure of the subject, and a calculation part that calculates an electrical characteristic of a region including the tissue structure using measurement data obtained by measurement of the region performed by the measurement part, wherein the calculation part comprises an electrical characteristic calculation part that calculates a rotating magnetic field from the measurement data, and calculates the electrical characteristic using the rotating magnetic field, the measurement data are measurement data measured by setting the direction of the tissue structure to be a predetermined direction of a coordinate system of the apparatus, and the electrical characteristic calculation part calculates the electrical characteristic including anisotropy using a relation between the direction of the tissue structure and the predetermined direction in the coordinate system of the apparatus stored in the storage part.

The electrical characteristic measuring method of the present invention is an electrical characteristic measuring method comprising measuring an electrical characteristic of a predetermined region of a subject placed in a static magnetic field space by using magnetic resonance signals measured from the region, wherein a rotating magnetic field map of the region is created from measurement data consisting of the magnetic resonance signals, the electrical characteristic is calculated by using the rotating magnetic field map, and in this calculation, the electrical characteristic is calculated as electrical characteristic including anisotropy by using the measurement data measured for two or more kinds of different arrangements of the subject that provide different directions of the subject with respect to the direction of the static magnetic field.

### Effect of the Invention

According to the present invention, an electrical characteristic such as electrical conductivity including anisotropy can be measured with good accuracy.

### BRIEF DESCRIPTION OF THE DRAWINGS

FIG. 1 shows outline of the whole body of an MRI apparatus, which is used as the electrical characteristic measuring apparatus.

FIG. 2 is a functional block diagram showing the configuration of the calculation part of the electrical characteristic measuring apparatus.

FIG. 3 comprises drawings for explaining simulation for obtaining an axis for which an electrical characteristic is maximized, FIG. 3(a) shows the relation between a phantom and an RF irradiation coil used for the simulation, and FIG. 3(b) is a table showing the set electrical conductivities of the phantom.

FIGS. 4(a) to 4(d) are photographs showing the results of the simulation.

FIG. 5 is a diagram showing the procedure of the electrical characteristic measuring method.

FIGS. 6(a) to 6(c) are drawings showing examples of anisotropy model of electrical conductivity.

FIG. 7 is a diagram showing the procedure of the electrical characteristic measuring method of the first embodiment.

FIG. 8 is a drawing showing relation of a tissue (nerve fiber) and an anisotropy model of electrical conductivity.

FIGS. 9(a-1) and 9(b-1) are drawings showing positions at the time of the electrical characteristic measurement, and FIGS. 9(a-2) and 9(b-2) are drawings showing the axes of the electrical conductivity that enable the most accurate measurement for the positions 1 and 2, respectively.

FIGS. 10(a) and 10(b) show examples of GUI for setting a subject.

FIGS. 11(a) and 11(b) show examples of display of electrical conductivity in the electrical characteristic measuring apparatus of the first embodiment.

FIG. 12 is a diagram showing the procedure of the electrical characteristic measuring method of the second embodiment.

FIG. 13 is a drawing for explaining correction of electrical conductivity for the main axis direction according to the second embodiment.

FIG. 14 is a drawing for explaining correction of electrical conductivity for a direction crossing the main axis direction according to the second embodiment.

FIG. 15 is a functional block diagram showing the configuration of the calculation part of the electrical characteristic measuring apparatus of the fourth embodiment.

### MODES FOR CARRYING OUT THE INVENTION

Hereafter, embodiments of the present invention will be explained with reference to the drawings.

### <Configuration of Apparatus>

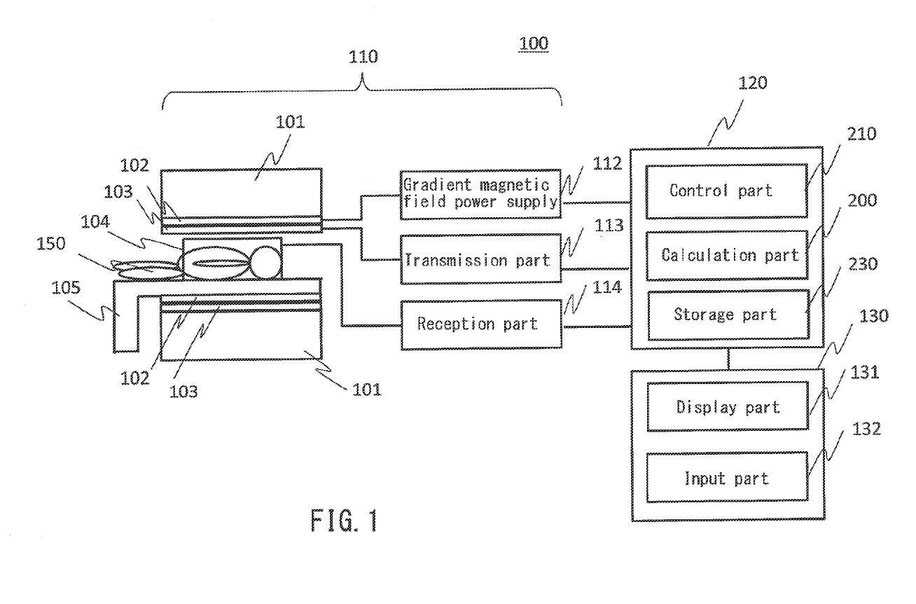

First, the outline of the electrical characteristic measuring apparatus will be explained with reference to the block diagram of FIG. 1. This electrical characteristic measuring apparatus 100 basically has the same configuration as that of a magnetic resonance imaging (MRI) apparatus, and according to general classification, it comprises a measurement part 110 provided with a static magnetic field generator 101, and so forth, a signal processing part 120, and an operation part 130.

The measurement part 110 comprises a static magnetic field generator 101 that generates a uniform static magnetic field in a space in which a subject 150 is placed, an RF irradiation coil 102 that is disposed so that it surrounds the subject in the static magnetic field space and transmits radio frequency waves (electromagnetic waves) for exciting nuclear spins in a tissue constituting the subject, a gradient coil 103 that imparts a magnetic field gradient to the static magnetic field, and an RF probe 104 that receives magnetic resonance signals (NMR signals) emitted from the subject in response to the electromagnetic waves generated by the RF coil 102. The subject 150 is placed in the static magnetic field space in a state of, for example, being laid down on a bed 105.

As the static magnetic field generator 101, there are generally known those of the horizontal magnetic field type, which generate a static magnetic field in the horizontal direction, and those of the vertical magnetic field type, which generate a static magnetic field in the vertical direction, and those in which the direction of the static magnetic field is inclined to the direction of the body axis of the subject may also be contemplated. Any of those types can be used for the present invention. As also for the method for generating a static magnetic field, there are those of permanent magnet type, normal conduction magnet type, and super-conductive magnet type, and any of these may be used.

The gradient coil 103 is for imparting different phase rotations to the NMR signals depending on the positions so as to impart positional information, and usually consists of a set of gradient coils for three axial directions of X, Y, and Z. These gradient coils are connected to a gradient magnetic field power supply 112, and can generate a gradient magnetic field for an arbitrary direction by changing the ratio of the electric currents for driving them for the three axes.

The RF irradiation coil 102 is for generating radio frequency signals of a frequency adjusted to the magnetic resonance frequency of the nuclear spin as the object of the measurement, and is connected to a transmission part 113 comprising a radio frequency oscillator that generates radio frequency waves of the magnetic resonance frequency, a radio frequency amplifier, and so forth. A radio frequency signal is usually applied to the subject as a pulse (radio frequency signal that is generated by the RF irradiation coil 102 is henceforth referred to as RF pulse). In common MRI apparatuses, the object of the measurement is hydrogen nuclei, and the magnetic resonance frequency is adjusted to the magnetic resonance frequency of proton. However, the object of the measurement is not limited to hydrogen, and the magnetic resonance frequency may be adjusted to a magnetic resonance frequency of another nuclide.

The RF probe 104 is an antenna adjusted so that it can receive the magnetic resonance signals, which are radio frequency signals, and is disposed near the subject 150. The RF probe 104 is connected to a reception part 114 comprising an amplifier, quadrature detection circuit, analog to digital converter, and so forth, and the NMR signals detected by the RF probe 104 are sent to a signal processing part 120 via the reception part 114, and used here for image reconstruction and other various calculations. The RF irradiation coil 102 may also serve as the RF probe 104, and in such a case, the function of the coil is alternately switched between those of the transmission part 113 and reception part 114 to transmit radio frequency signals and receive NMR signals.

The signal processing part 120 mainly consists of a CPU and a memory, and an external storage (not shown in the drawing), and the operation part 130 are connected to it. In this specification, the internal memory of the signal processing part 120 and external storage are collectively called storage part 230. The signal processing part 120 comprises a calculation part 200 that performs calculations of image reconstruction, image processing, correction, and so forth using the NMR signals sent from the reception part 114 mentioned above, and a control part 210 that controls the operations of the measurement part 110 and the calculation part 200. A pulse sequence that determines timings of applications of the RF pulses and gradient magnetic field pulses by the measurement part 110, timings of measurement of NMR signals, and so forth, is included in the control part 210 as a program. As the pulse sequence, various sequences corresponding to various imaging methods are known, and the control part 210 controls the operation of the measurement part 110 according to a pulse sequence corresponding to the imaging method used.

The processings carried out by the calculation part 200 include calculation of electrical characteristic using NMR signals, creation of electrical characteristic image, correction processing performed as required, and so forth. Although typical examples of the electrical characteristic are electrical conductivity and permittivity, the electrical characteristic is not be limited to these, and may also be any of various amounts drawn from them. The calculations and processings performed in the calculation part 200 are realized by executing a program loaded on CPU beforehand. However, a part of the processings may be realized by such hardware as ASIC (Application Specific Integrated Circuit) and FPGA (Field-programmable Gate Array).

The operation part 130 is for setting conditions required for processings performed by the control part 210 or the calculation part 200, and displaying GUI and processing results, and comprises an input part 132 having an input device such as keyboard and mouse, and a display part 131 comprising CRT, liquid crystal panel, or the like.

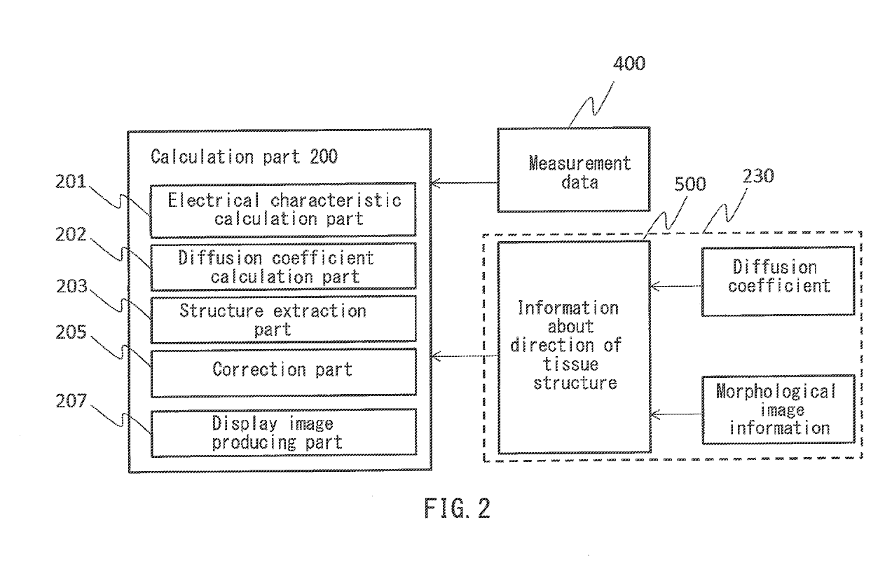

On the basis of the above explanations of the configuration, the outline of the electrical characteristic measurement using the electrical characteristic measuring apparatus of the present invention will be explained. First, the configuration of the calculation part 200 and the data that are used in the calculation part 200 will be explained with reference to FIG. 2. The diagram of FIG. 2 mentions elements that can be provided in the calculation part 200 corresponding to any of the embodiments to be explained below in detail, and include those that can be omitted depending on the embodiment. An element that is not shown in FIG. 2 may also be added.

As shown in the diagram, the calculation part 200 comprises the electrical characteristic calculation part 201. Measurement data 400 obtained with a pulse sequence executed by the measurement part 110 for the purpose of measurement of an electrical characteristic and information 500 about direction of tissue structure of a subject stored in the storage part 230 are inputted into the calculation part 200. The information about direction of tissue structure is information about the longitudinal direction of the tissue structure, and it may be an angle to a predetermined base direction, for example, the direction of the body axis, or a direction determined with a predetermined point (end or center) of the tissue structure itself as the base. Typical examples are, in the case of fiber structure, the direction of laying fibers, and in the case of blood vessel, the running direction thereof.

The method for obtaining the information 500 about direction stored in the storage part 230 is not particularly limited. The information may be one obtained with an imaging apparatus or the like other than the MRI apparatus, or as information obtained with the MRI apparatus, one obtained from a morphological image, diffusion coefficient obtained from a diffusion-weighted imaging, and so forth can also be utilized. In the case of an electrical characteristic measuring apparatus that utilizes the diffusion coefficient, the calculation part 200 comprises a diffusion coefficient calculation part 202 that calculates diffusion coefficient using measurement data obtained by diffusion-weighted imaging. When a morphological image obtained with the MRI apparatus (proton density image, T1 or T2-weighted image, etc.) is used, the calculation part 200 preferably comprises a structure extraction part 203 that extracts tissue structure by using the image data.

The measurement data 400 are data measured under the conditions that the direction of tissue structure corresponds to the direction of the static magnetic field of the apparatus, or has a predetermined angle with respect to the direction of the static magnetic field, and may include a plurality of sets of measurement data consisting of those obtained with different angles to the direction of the static magnetic field. The imaging method for obtaining the measurement data 400 (pulse sequence) is not particularly limited, and for example, a pulse sequence of GE (gradient echo) type, which enables high-speed 3D data measurement, can be used.

The calculation part 200 can further comprise a correction part 205 that corrects the electrical conductivity calculated by the electrical characteristic calculation part 201, if needed, a display image producing part 207 for displaying UI (user interface) or displaying the electrical conductivity as a map or image, and so forth.

The electrical characteristic measurement using the electrical characteristic measuring apparatus (MRI) of the configuration explained above will be explained below.

First, the relation between anisotropy of an electrical characteristic and axes of coordinates of the electrical characteristic measuring apparatus, especially the direction of the static magnetic field, will be explained as a premise. This relation is found by such imaging or simulation as explained below.

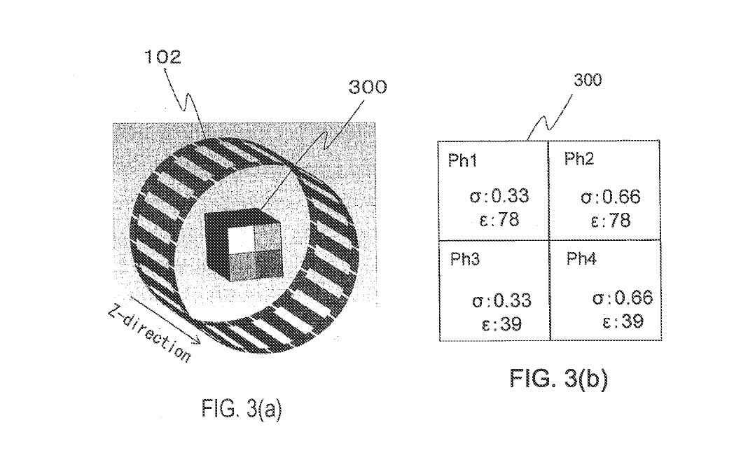

A phantom showing an electrical characteristic including anisotropy is prepared. For example, there is prepared a phantom 300 in the shape of rectangular parallelepiped, which consists of a combination of four homogeneous pillar-shaped phantoms Ph1 to Ph4, as shown in FIG. 3(a). The values of permittivity of the phantoms are set so that, as shown in the table of FIG. 3(b), the values of permittivity of the phantoms adjacent to each other for the y-direction are the same, and those of the phantoms adjacent to each other for the x-direction are different. The electrical conductivities of the phantoms are set so that, as shown in the table of FIG. 3(b), the electrical conductivities of the phantoms adjacent to each other for the x-direction are the same, and those of the phantoms adjacent to each other for the y-direction are different. Further, the electrical conductivity of the upper right phantom Ph2 shown in the drawing is set so that the electrical conductivity for only one of the x, y, and z-directions is different from those for the other directions, and thereby set to be anisotropic electrical conductivity.

Such a phantom 300 is set, and electrical conductivity is calculated for every pixel by simulation. In the simulation, a rotating magnetic field is calculated by setting an RF irradiation pulse of the nuclear magnetic resonance frequency, and electrical conductivity is calculated for every pixel.

Electrical conductivity and permittivity can be calculated from the calculated rotating magnetic field (H⁺) in accordance with the following equations (1) and (2) as electrical conductivity σ and permittivity ε.

$$\begin{matrix}[ Equation 1 ] & \\ σ = \frac{1}{ϖ {μ}_{0}} Im [ \frac{{▽}^{2} {H}^{+} ( r )}{{H}^{+} ( r )} ] & ( 1 ) \\ ɛ = \frac{- 1}{{ϖ}^{2} {μ}_{0}} Re [ \frac{{▽}^{2} {H}^{+} ( r )}{{H}^{+} ( r )} ] & ( 2 )\end{matrix}$$

In the equation (1) and (2), ω is angular frequency (nuclear magnetic resonance frequency), and μ₀ is vacuous magnetic permeability, which is a known value. H⁺ is the measured rotating magnetic field, and r is coordinate of pixel.

By calculating electrical characteristic for various directions (axes) for which the electrical conductivity of the phantom Ph2 is changed, a direction (axis) for which the electrical characteristic can be most accurately measured is examined.

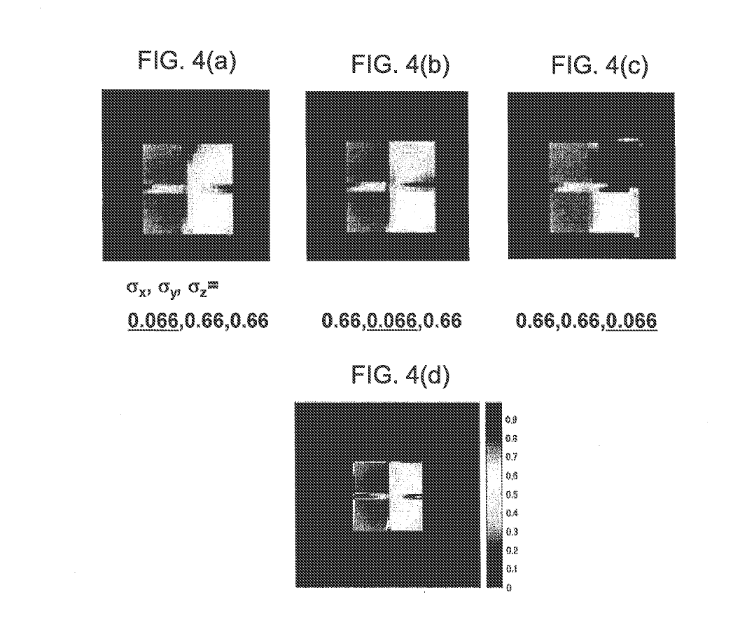

An electrical conductivity map obtained by calculating electrical conductivity through the simulation using the phantom exemplified in FIG. 3 is shown in FIG. 4. In FIG. 4, the direction perpendicular to the photographs is the direction of the static magnetic field (z-direction), and the photographs show the results of the simulation performed by using an electrical conductivity of the upper right phantom Ph2 for only one of the x, y, and z-directions that is 1/10 of the electrical conductivity for the other axes. The results shown in FIGS. 4(a) to 4(c) were obtained with electrical conductivities for the x, y, and z-directions σx, σy, and σz corresponding to 1/10 of the electrical conductivities for the other directions, respectively. The result shown in FIG. 4(d) was obtained with the four phantoms all of which showed isotropic electrical characteristic. As seen from these results, the electrical conductivity for the z-direction became small only in the case of FIG. 4(c) where the electrical conductivity for the z-direction was 1/10, and it can be seen that the accuracy of the electrical conductivity for the z-direction is high. That is, in this example, it was confirmed that, when the anisotropy of the electrical conductivity was taken into consideration, the electrical conductivity σz for the same direction as the direction of the static magnetic field can be most accurately measured. Although the direction that enabled the most accurate measurement was the same direction as the direction of the static magnetic field in this simulation, it is not limited to the direction of the static magnetic field. The direction can be determined by, for example, when relation between the electrical conductivity and the direction for which electrical conductivity is changed is obtained, using a smaller changing unit of the direction for which the electrical conductivity is changed.

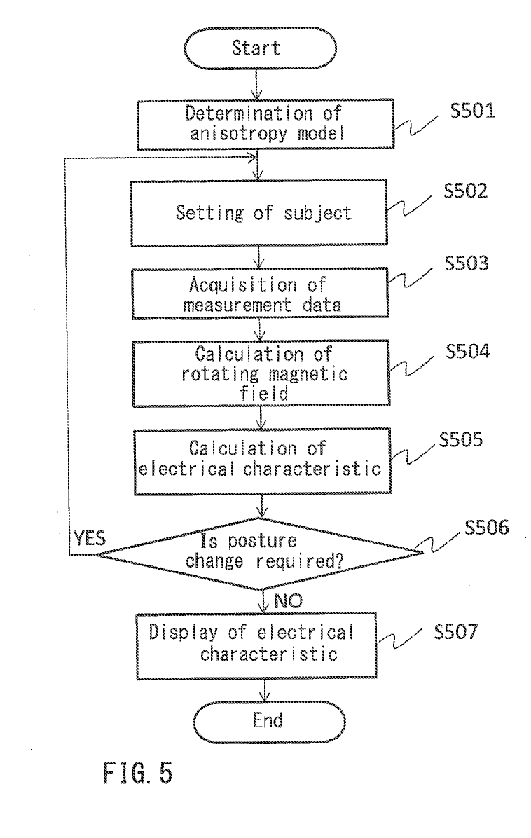

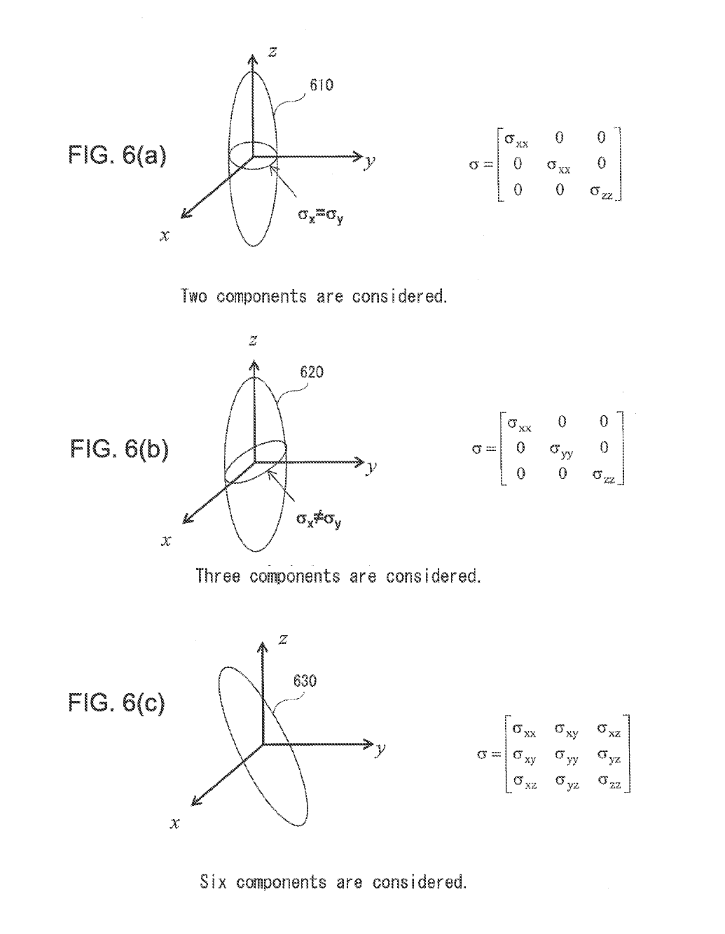

On the premise of the relation between the anisotropy of electrical characteristic and coordinate axes of the apparatus explained above, the method for measuring electrical characteristic measurement of the present invention is shown in FIG. 5. First, an anisotropy model of an electrical characteristic is determined for a part of the subject 150 as a measurement object using information on tissue structure relevant to the anisotropy of electrical characteristic (S501). As the anisotropy model, there are, as shown in FIGS. 6(a) to 6(c), a model 610 in which two components are taken into consideration, model 620 in which three components are taken into consideration, model 630 in which six components are taken into consideration and so forth. The model 610 is a model in which, for example, the electrical conductivity σz for the z-direction is largest, and those for the x and y-directions are isotropic. The model 620 is a model in which the electrical conductivity σz for the z-direction is largest, and those for the x and y-directions are also anisotropic (σx≠σy), and in the example shown in the drawing, σx is larger than σy (σx>σy). The model 630 is a generalized model, in which the xy-axis, yz-axis, and xz-axis are added to the x-axis, y-axis, and z-axis. Any of these anisotropy models may be used, and a predetermined model may be set beforehand. An anisotropy model may also be set by using spherical surface harmonics. The electrical characteristic calculation part 201 fits an anisotropy model to tissue structures stored in the storage part 230.

Then, a subject is placed in a static magnetic field space so that a predetermined axis of the anisotropy model should correspond to a predetermined direction in the coordinate system of the apparatus (S502). For example, a part of the subject including the tissue structure is placed so that the direction of the tissue structure along the z-axis of the anisotropy model should correspond to the direction of the static magnetic field of the electrical characteristic measuring apparatus. Then, NMR signals are measured with the measurement part 110 (S503). The NMR signals are measured in such a number that at least one image can be reconstructed to obtain k-space data (measurement data). The calculation part 200 performs Fourier transform of the k-space data to obtain real space data.

Subsequently, the electrical characteristic calculation part 201 calculates a rotating magnetic field (H⁺) from the signal values of the pixels of the real space data (image data) (S504). The rotating magnetic field around the z-axis (direction of the static magnetic field) may be H⁺ and H. However, when hydrogen nuclei are the measurement object, what contributes to the nuclear magnetic resonance phenomenon is H⁺, and therefore H⁺ is calculated in this case. As the method for calculating H⁺, there are the method of obtaining difference of an image obtained by using an RF pulse of an arbitrary flip angle and an image obtained by using an RF pulse of a flip angle being twice of the foregoing arbitrary flip angle so as to calculate absolute value of H⁺ (double angle method), the method of performing a plurality of times of measurement using a pre-pulse with different times T1 from the application of the pre-pulse, and calculating absolute value of B1 from image data obtained with different TI values (for example, the method described in Republication of WO2012/060192), and so forth, and any of those may be used. The phase of H⁺ can be estimated by using, for example, phase information of image. Since signal intensity of each pixel (complex number) S(r) can be approximately represented by the equation (3), the rotating magnetic field H⁺ may be calculated by solving the equation (3) using signal intensities of a plurality of images obtained with different flip angles.

[Equation 3]

S(r)≈M₀(r)sin(c·H⁺(r)) (3)

In the equation, M₀ represents longitudinal magnetization, and c is an apparatus-specific constant.

When H⁺ is calculated, such a treatment as masking may be performed for portions where the subject does not exist, or portions other than the objective tissue structure. Then, from the rotating magnetic field H⁺, the electrical conductivity σ and permittivity ε are calculated by using the equations (1) and (2) mentioned above (S505).

Similar measurements are repeated as required with changing the direction of the tissue as the object of the measurement from that for the first measurement (S506). The electrical characteristic finally calculated in S505 is displayed on the display part 131 (S507).

According to the present invention, by performing imaging with the direction of the tissue structure relevant to electrical characteristic anisotropy set to correspond to a direction that enables the most accurate measurement of the electrical characteristic, for example, the direction of the static magnetic field, electrical characteristic including anisotropy can be measured with good precision. By performing at least two times of measurement with different directions of the tissue structure, detailed information on the anisotropy can be obtained.

Hereafter, specific embodiments of the electrical characteristic measurement using the electrical characteristic measuring apparatus of the present invention will be explained. The following embodiments will be explained for examples of calculating electrical conductivity among electrical characteristics.

### First Embodiment

In the electrical characteristic measuring apparatus of this embodiment, diffusion coefficient data measured beforehand for a subject as an object of the electrical characteristic measurement are stored in the storage part 230, and the calculation part 200 obtains information about the axis for which the diffusion coefficient is maximized in the tissue structure, and determines an anisotropy model of the electrical characteristic. The calculation part 200 calculates the electrical characteristic using a plurality of sets of measurement data obtained by performing the measurement with at least two kinds of different arrangements of the subject. The arrangements of the subject are determined in consideration of the relation of the axis for which the diffusion coefficient is maximized and the direction of the static magnetic field, and an anisotropy model of the electrical characteristic.

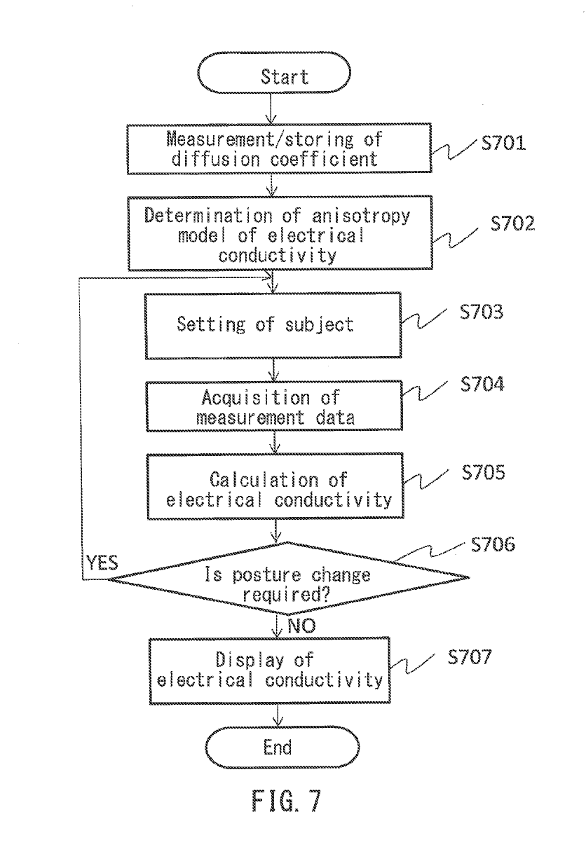

Hereafter, the procedure of the measurement method using the electrical characteristic measuring apparatus of this embodiment will be explained with reference to FIG. 7.

First, diffusion coefficient is measured, and results are stored in the storage part 230 (S701). The method for measuring diffusion coefficient is the same as the known measurement method utilizing an MRI apparatus. Briefly, for example, a diffusion-weighted pulse sequence such as ss-DWEPI (single shot Diffusion-Weighted Echo Planar Imaging) including application of an MPG (Motion Probing Gradient) pulse is executed to collect magnetic resonance signals. In this step, the measurement is performed a plurality of times with different application directions m of the MPG pulse and b values as an index of the intensity of the pulse, and the diffusion coefficient is calculated from signal values S(m, b) of the pixels of the obtained diffusion-weighted image in accordance with, for example, the following equation (function of the diffusion coefficient calculation part 202).

$$\begin{matrix}[ Equation 4 ] & \\ S ( m , b ) = {S}_{0} \cdot Exp ( - b \cdot {ADC}_{m} + \frac{1}{6} {b}^{2} \cdot {ADC}_{m}^{2} \cdot {AKC}_{m} ) & \end{matrix}$$

In the equation, S₀ is the signal intensity when b value is 0 (=S(m, 0)), and ADCₘ is the diffusion coefficient for the application direction m of the MPG pulse. Although AKCₘ is a kurtosis coefficient of the application direction m, and it is an unknown in the equation, it can be eliminated by solving a plurality of equations. For the calculation method, the quasi-Newton method, nonlinear least square fitting without constraint such as the Levenberg-Marquardt method, and so forth are used. The calculated diffusion coefficients for every pixel are stored in the storage part 230.

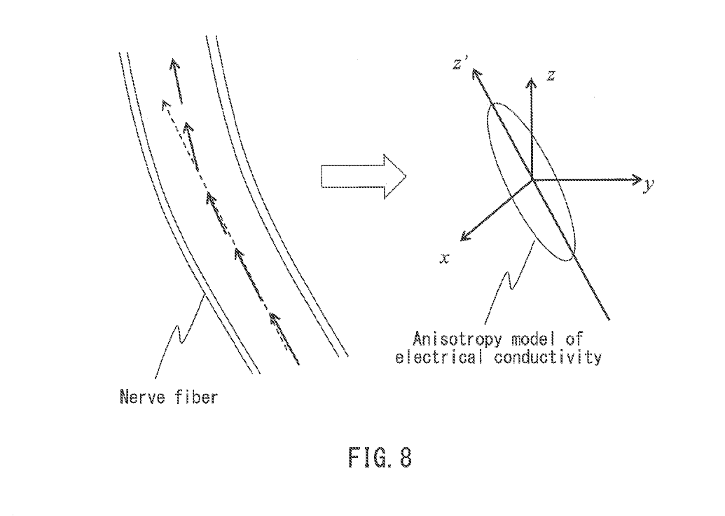

In general, tissue structures of muscular fiber, nerve fiber etc. along the major axis direction and the minor axis direction of the fibers are different, and show different diffusion coefficients due to such difference in the structure. It can also be estimated that electrical characteristics should be different depending on the tissue structure, i.e., electrical characteristics along the major axis direction and the minor axis direction should be different. In this embodiment, such an anisotropy model 610 as shown in FIG. 6A, which show the maximum electrical conductivity for the axial direction (main axis direction) for which it also show the maximum diffusion coefficient, and isotropic diffusion coefficient for the direction perpendicular to the direction of the main axis, is used as an anisotropy model of electrical conductivity, and it is fit to tissue structure (S702). FIG. 8 is a drawing showing fitting of the anisotropy model of electrical conductivity to a nerve fiber. The direction of the main axis of the diffusion coefficient may be determined from an average value of the diffusion coefficients of the pixels in a predetermined area, or may be determined from the diffusion coefficient of the pixel located at the center of a predetermined area.

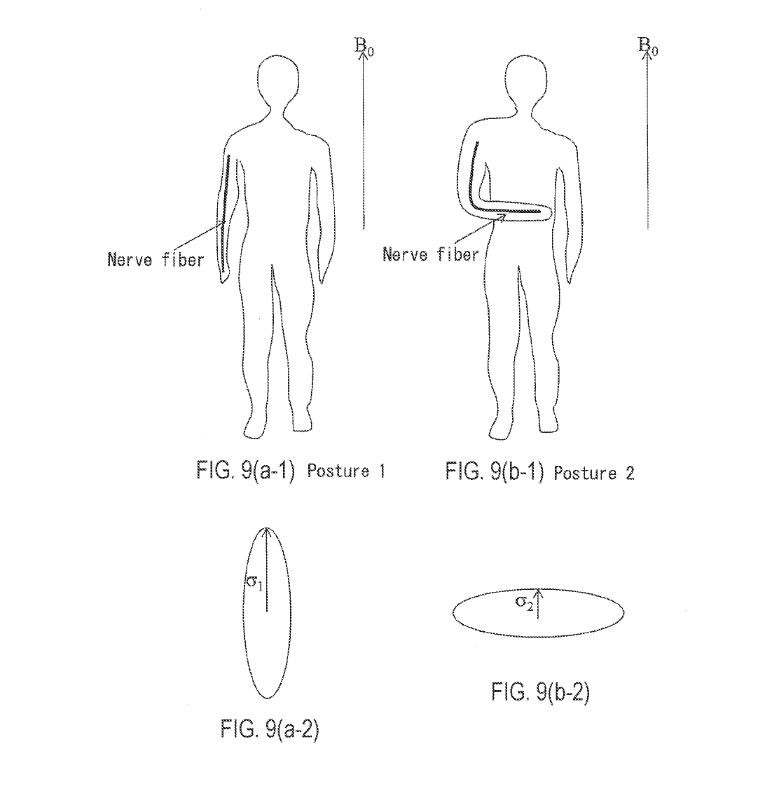

Then, electrical characteristic measurement is performed (S703 to S706). The measurement is performed at least twice with different postures of the objective part of the subject. Examples of the different positions are shown in FIG. 9. In these examples, nerve fibers of the upper extremity of the subject are the measurement object, and the axial direction for which maximum diffusion coefficient is obtained (=main axis direction) is the direction along the nerve fibers. In the posture 1, the direction of the main axis (longitudinal direction of the nerve fibers) is coincided to the direction of the static magnetic field as shown in FIG. 9(a-1), and in the posture 2, the direction of the main axis is coincided to the direction perpendicular to the direction of the static magnetic field as shown in FIG. 9(b-1). The direction of the static magnetic field is the direction found as the direction that allows the most accurate measurement of the electrical characteristic by simulation. FIGS. 9(a-2) and 9(b-2) are drawings showing anisotropy models corresponding to the postures 1 and 2, respectively.

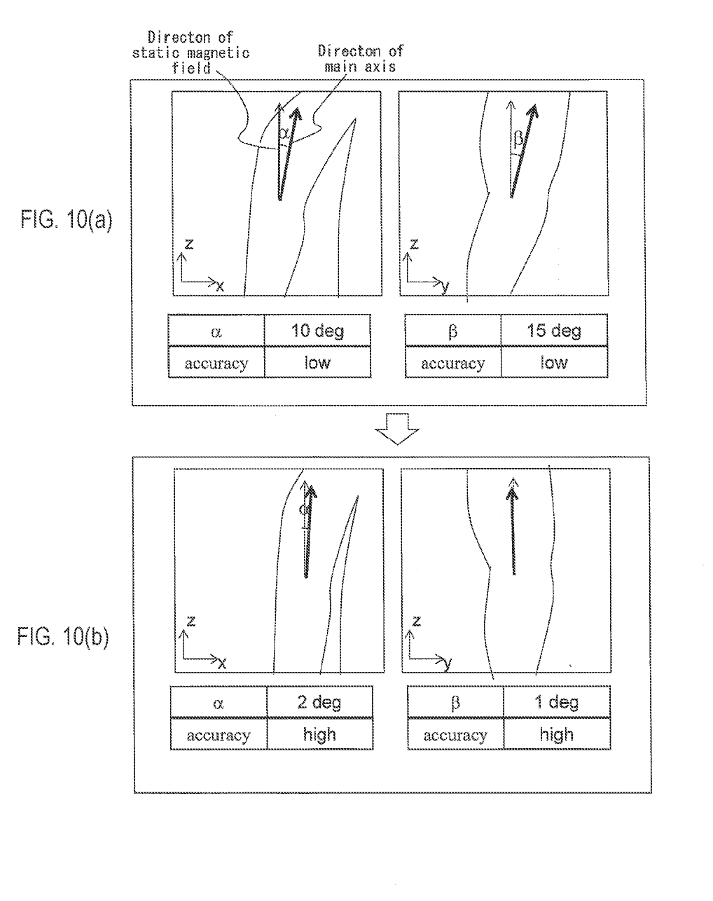

In order to accurately place the subject in such positions, the display image producing part 207 may create GUI for supporting the positioning, and display it on the display part 131. An example of GUI for accurately coinciding the direction of the tissue structure of the subject to a predetermined direction (direction of static magnetic field) is shown in FIG. 10. FIG. 10(a) shows a screen displaying images acquired for the positioning, in which the acquired images are superimposed on images of a part as the object of imaging, and the direction of the main axis of the diffusion coefficient stored in the storage part 230 is displayed with an arrow (vector) or the like. In FIG. 10, images of two sections including the direction of the static magnetic field (z-direction), zx-plane and zy-plane, are displayed, and the values of angles α and β between the direction of the static magnetic field and the direction of the main axis, and coincidence degrees (accuracies) with respect to the direction of the static magnetic field in the sections are displayed below the images as tables. The coincidence degree may be displayed with a qualitative expression of “high”, “low”, or the like defined on the basis of a predetermined threshold. In the example shown in the drawing, if they coincide with a difference within several degrees, the indication of “high” is displayed (FIG. 10(b)), and if the difference is larger than that, the indication of “low” is displayed (FIG. 10(a)). On the basis of such information displayed by GUI, an operator arranges or rearranges the subject in the static magnetic field space (S703), and determines whether measurement (main imaging) for measuring electrical characteristic is performed, or a positioning image is obtained again.

In the measurement of electrical characteristic, although the pulse sequence is not particularly limited, a GE type pulse sequence of 2D or 3D, for example, is executed, and measurement data of 2D or 3D are collected (S704). Then, the electrical characteristic calculation part 201 calculates the electrical characteristic by using the measurement data (k-space data) obtained by the execution of such a pulse sequence (S705). For this purpose, Fourier transform of the k-space data obtained with the posture 1 is first carried out to obtain real space data. From the signal values (complex numbers) of the pixels of the real space data, a rotating magnetic field H⁺ is calculated, for example, in accordance with the equation (3). Subsequently, from the rotating magnetic field H⁺, electrical conductivity σ is calculated by using the equations (1) and (2) mentioned above. For the model 610 in which electrical conductivity is maximized for the z-axis direction (σz), and isotropic for the x-axis and y-axis directions perpendicular to the z-axis direction, electrical conductivity can be described with the tensor represented by the following equation (5).

$$\begin{matrix}[ Equation 5 ] & \\ σ = ( \begin{matrix}{σ}_{xx} & 0 & 0 \\ 0 & {σ}_{xx} & 0 \\ 0 & 0 & {σ}_{zz}\end{matrix} ) & ( 5 )\end{matrix}$$

In the measurement with the posture 1, the axis for which the electrical conductivity is maximized and the direction of the static magnetic field are coincided, and therefore the value σ1 of the maximum electrical conductivity can be accurately measured.

Similar calculation is performed for the measurement data obtained with the posture 2 to calculate electrical conductivity. Electrical conductivity σ2 for the direction of the axis perpendicular to the direction of the axis for which electrical conductivity is maximized can be thereby obtained. The values of electrical conductivity obtained with the two kinds of postures, σ1 and σ2, are equal to each other (σ1=σ2) in a tissue structure where electrical conductivity is isotropic, but different in a nerve fiber (σl≠σ2, σ1>σ2 in the example mentioned above), and thus information on electrical conductivity including anisotropy can be obtained.

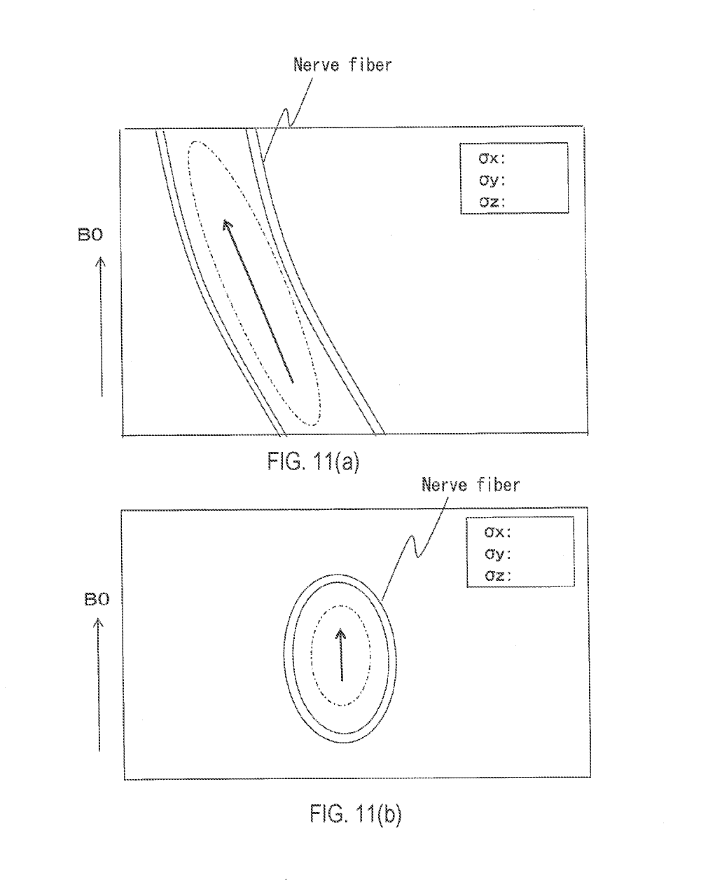

The display image producing part 207 creates an image to be displayed on the display part 131 by using the electrical conductivity obtained in such a manner as described above (S707). Although the display scheme is not particularly limited, for example, anisotropy can be displayed with a vector or as an ellipse indicating an anisotropy model on a separately obtained image of a tissue structure or outline image thereof, and values of electrical conductivity can also be displayed together as a table, or the like, as shown in FIG. 11.

As explained above, in the electrical characteristic measuring apparatus of this embodiment, the calculation part 200 calculates electrical characteristic of a region including a tissue structure by using first measurement data obtained by performing measurement of the region with setting the direction of the tissue structure to be a first direction in the coordinate system of the apparatus, and second measurement data obtained by performing measurement of the region with setting the direction of the tissue structure to be a second direction in the coordinate system of the apparatus. In this embodiment, the information about the direction of the tissue structure stored in the storage part consists of diffusion coefficient obtained by the measurement part through measurement of the region including the tissue structure.

According to this embodiment, by performing the measurement with two positions, in which the main axis directions in the coordinate of the electrical characteristic measurement apparatus are different, using information about anisotropy of the electrical characteristic obtained separately from the electrical characteristic measurement and an anisotropy model, and measuring the electrical characteristic using the measurement data, the electrical characteristic including anisotropy can be measured with good accuracy.

In the above explanation of this embodiment, the anisotropy model 610 shown in FIG. 6(a), in which the electrical characteristic is isotropic for the x-direction and y-direction, is determined as the anisotropy model. However, it is also possible to determine such a model 620 that shows anisotropy also for the xy-plane (model determined in consideration of three components) as shown in FIG. 6(b) as the anisotropy model. As such a model, for example, a tissue structure of a shape having a flat section for the direction perpendicular to the longitudinal direction can be contemplated, and an anisotropy model can be determined from that shape. In this case, by performing the measurement with the posture 2 in which the direction of the main axis coincides to a first direction perpendicular to the direction of the static magnetic field, and the posture 3 in which the direction of the main axis coincides to a second direction perpendicular to the direction of the static magnetic field and the first direction, in addition to the posture 1 in which the direction of the main axis coincides to the direction of the static magnetic field, information about anisotropy including three components can be obtained. Further, such a model determined in consideration of six components as shown in FIG. 6(c) may be set, and the measurement may be performed with positions for six directions. When a model using spherical surface harmonics is set, the measurement may be performed for a direction of a variable of the spherical surface harmonics used.

### Second Embodiment

In the electrical characteristic measuring apparatus of the first embodiment, one direction is supposed as the main axis direction for which the diffusion coefficient is maximized, and the measurement of the electrical characteristic is performed with coinciding this direction to the direction of the static magnetic field. In this embodiment, a function of correcting the measurement results when the direction of the main axis changes for every pixel is added as a function of the calculation part 200, so that a non-linear tissue structure can be dealt with. That is, the calculation part 200 of the electrical characteristic measuring apparatus according to this modification comprises the correction part 205 (FIG. 2).

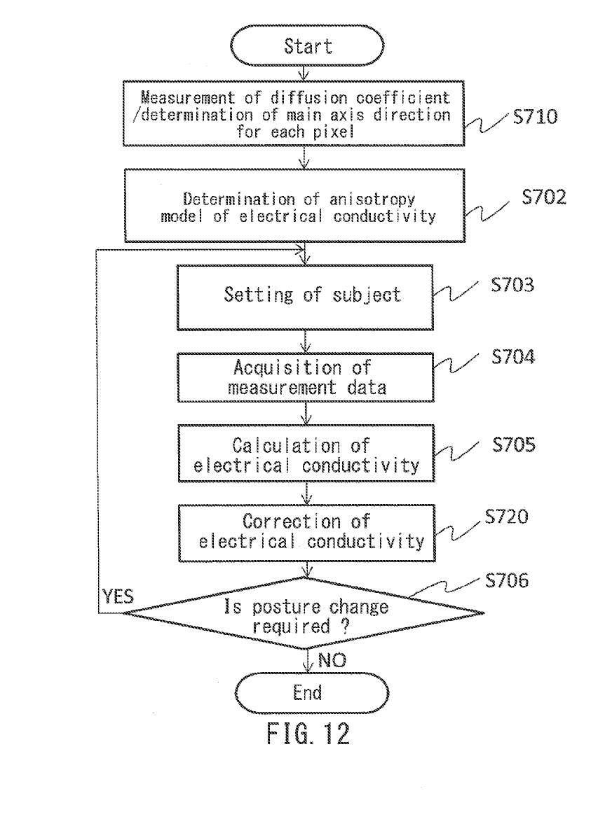

Hereafter, the operation of the apparatus of this embodiment will be explained mainly for the characteristics different from those of the first embodiment. The flow of the processings is shown in FIG. 12. In FIG. 12, the same processings as those shown in FIG. 7 are indicated with the same numerals, and detailed explanations thereof are omitted.

First, data for calculating diffusion coefficient are measured, direction of the main axis for which the diffusion coefficient is maximized is determined (S710), and an anisotropy model of electrical conductivity is determined (S702). In S710, the main axis is determined for every pixel, and a main axis direction that serves as the basis for determining the anisotropy model in S702 is chosen. The main axis direction that serves as the basis may be determined as an average or median for a predetermined range like the main axis direction used in the first embodiment.

Subsequently, when the anisotropy model is such an anisotropy model 610 as shown in FIG. 6(a), measurement for electrical characteristic measurement is performed with the posture 1 in which the main axis direction coincides to the direction of the static magnetic field, and the posture 2 in which the main axis direction is perpendicular to the direction of the static magnetic field (S703, S704), and rotating magnetic field and electrical conductivity are calculated (S705).

Then, the correction part 205 calculates electrical conductivity σ1 that is the maximum value of the eigenvalues for every pixel by using electrical conductivity (measured electrical conductivity) σ_{z} calculated for each pixel and angle θ between the main axis direction and the direction of the static magnetic field for each pixel determined in S710 in accordance with the following equation (6) (S720).

$$\begin{matrix}[ Equation 6 ] & \\ {σ}_{1} = \frac{{σ}_{z}}{cos θ} & ( 6 )\end{matrix}$$

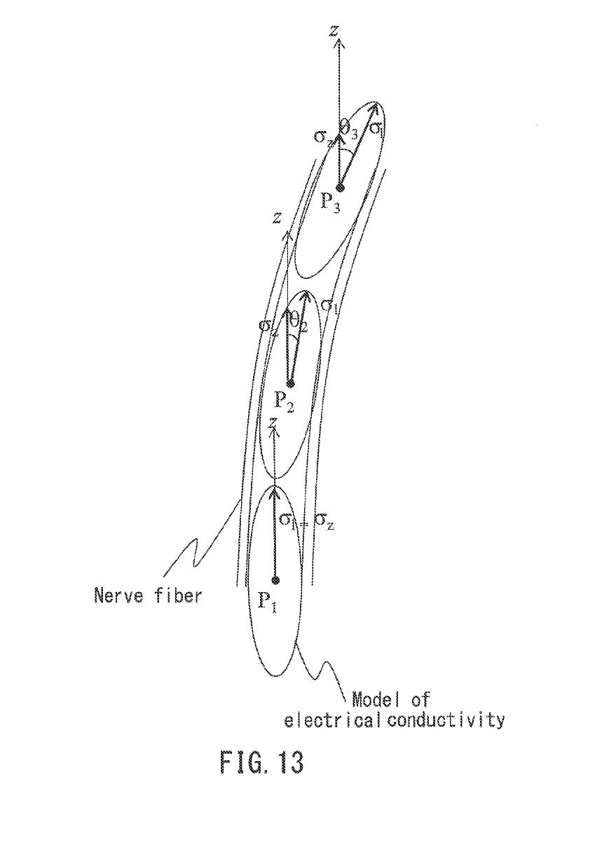

An explanatory drawing of the processing performed by this correction part 205 is shown in FIG. 13. As shown in FIG. 13, in a tissue generally extending along the z-direction, but mildly tuning (for example, nerve fiber), if an anisotropy model of electrical conductivity is determined for each of the points (pixels) P1 to P3, the main axis direction coincides to the direction of the static magnetic field (z-direction) at P1. If the measurement is performed with the main axis direction at P1 as the base main axis direction, there are angles θ2 and θ3 between the main axis directions and the direction of the static magnetic field (that is, the base main axis direction) at P2 and P3, respectively. In the measurement of S603, electrical conductivity σ_{z} is obtained as the most reliable and accurate value, and it can be regarded as the z-direction component of σ1 for the main axis direction. Therefore, by correcting σ_{z} through calculation of the equation (6) using θ2 and θ3 as θ, the maximum value of eigenvalues for P2 and P3 can be obtained. By performing this correction for all the pixels, σ1 can be calculated for all the pixels.

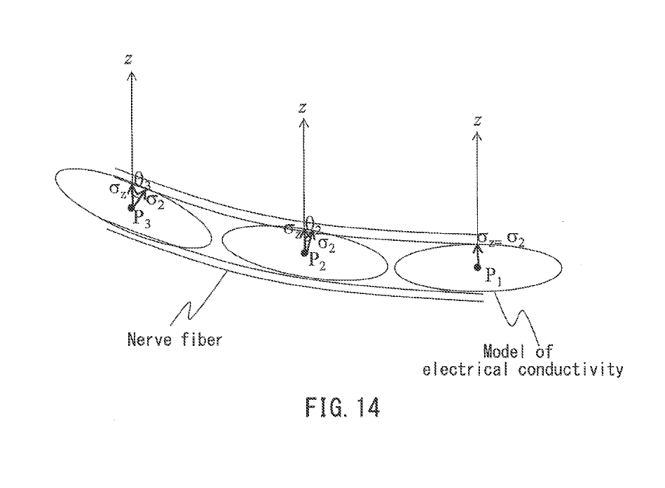

The same shall apply to the electrical conductivity σ2, which is measured with the main axis direction of the diffusion coefficient that coincides to the direction perpendicular to the direction of the static magnetic field. When the angles between the direction perpendicular to the main axis direction and the direction of the static magnetic field at P2 and P3 shown in FIG. 14 are θ2 and θ3, by correcting the electrical conductivity σ_{z} obtained by the measurement using these θ2 and θ3 in accordance with the equation (6), electrical conductivity σ2 for the direction perpendicular to the main axis direction can be calculated. By performing this correction for all the pixels, σ2 can be calculated for all the pixels, and by this collection and the processing shown in FIG. 11, σ1 and σ2 of all the pixels can be calculated.

It is the same as the first embodiment that the obtained electrical conductivities σ1 and σ2 may be then displayed on the display part 131 as a desired display image or numerical values.

The electrical characteristic measuring apparatus of this embodiment is characterized in that the calculation part further comprises a correction part that corrects the electrical characteristic calculated by the calculation part from a rotating magnetic field, by using an angle between the continuing direction of the tissue structure and the axis direction in the coordinate system in which the rotating magnetic field can be most accurately detected. According to this embodiment, electrical conductivity including anisotropy can be accurately obtained for all the pixels for the tissue structure as the object of the measurement.

### Modification of Second Embodiment

The aforementioned first embodiment is explained for a case where the main axis direction of the diffusion coefficient is coincided to the direction in which electrical characteristic can be most accurately measured (for example, the direction of the static magnetic field). However, if information about the angle between the main axis direction of the diffusion coefficient and a predetermined axial direction of the apparatus is obtained beforehand, the electrical conductivity for the main axis direction can be calculated by using the function of the correction part of the second embodiment even when the main axis direction does not coincide with the predetermined axial direction.

That is, in this modification, in the step S710 shown in FIG. 12, the main axis direction is determined beforehand for every pixel, and angle θa between the main axis direction of each pixel and the base main axis direction is also obtained beforehand. Next, a subject is placed so that direction of a tissue of the subject as the measurement object approximately coincides to, for example, the direction of the static magnetic field by using a positioning image. In this positioning image, the main axis direction of the diffusion coefficient is displayed as shown in FIG. 10. This main axis direction is the base main axis direction, and angle θb between this direction and the direction of the static magnetic field is also displayed. The angle θ between the main axis direction of each pixel and the direction of the static magnetic field can be obtained from the angle θb between the base main axis direction and the direction of the static magnetic field, and the angle θa between the main axis direction and the base main axis direction of each pixel.

The measurement of the electrical characteristic is performed with the position of the subject with which the positioning image used for obtaining θb is obtained, and electrical conductivity σz is calculated. By correcting this electrical conductivity in accordance with the equation (6) using the angle θ between the main axis direction and the direction of the static magnetic field of each pixel obtained beforehand, electrical conductivity σ1 for the axis in which electrical conductivity is maximized can be obtained. By performing the same procedure with another posture, for example, a posture in which the base main axis direction approximately coincides to the direction perpendicular to the direction of the static magnetic field, electrical conductivity σ2 can be obtained. Although not shown in FIG. 12, it is the same as the first and second embodiments that the obtained electrical conductivities may be displayed on the display part 131 in any of various display schemes.

According to this modification, electrical characteristic can be measured in the step in which the subject is placed in a desired posture without repeating replacement of the subject. The throughput of the measurement is thereby improved, and burdens imposed on the subject and operator can also be reduced.

### Third Embodiment

In the electrical characteristic measuring apparatus of the first embodiment, an anisotropy model of electrical conductivity is determined on the basis of information obtained from diffusion coefficient. However, in this embodiment, an anisotropy model of electrical characteristic is determined from a morphological image obtained beforehand. For this purpose, the calculation part 200 of the electrical characteristic measuring apparatus of this embodiment comprises a structure extraction part 203 (FIG. 2).

The procedures of the electrical characteristic measurement according to this embodiment other than those of S701 shown in FIG. 7 and S710 shown in FIG. 12 are the same as those of the first embodiment, the second embodiment, or the modification thereof. The operation of the electrical characteristic measuring apparatus of this embodiment will be explained with reference to FIG. 7 used for the explanation of the operation of the first embodiment, as required.

First, the measurement part 110 obtains an image of a region including a tissue of a subject as an object of the electrical characteristic measurement by imaging of the region. The imaging method is not particularly limited, so long as structure of the objective tissue can be grasped. When electrical characteristic of 3D is desired, 3D imaging is performed. Then, the structure extraction part 203 extracts a tissue from the image obtained by the imaging. The method for extracting a tissue is not particularly limited, and there can be used a method in which an operator specifies a contour of an objective tissue with looking at an image displayed on the display part 131, a method of obtaining T1-weighted image and T2-weighted image, and automatically extracting a tissue using the difference of the images, and so forth. Subsequently, the directions and lengths of the major and minor axes of the tissue are calculated. The information including these is stored in the storage part 230 as information concerning the tissue structure.

When it is desired to obtain the direction of the tissue for every pixel in this embodiment, for example, by extracting a line along the continuing direction of the tissue structure and determining tangential directions at a plurality of points on the line in the structure extraction part 203, directions at each point can be obtained.

It is the same as the first embodiment or second embodiment that an anisotropy model of electrical conductivity is then determined by using this information about the tissue structure, and measurement of the electrical characteristic is performed, in which correction according to the angle between the direction of the tissue and the direction of the static magnetic field may be performed as required.

According to this embodiment, the operation for diffusion coefficient is not required, and therefore information concerning tissue structure can be obtained in a comparatively short period of time. Further, since the major axis direction or minor axis direction of tissue structure is directly determined, it is unnecessary to newly calculate the angle θ between the direction of tissue and the direction of the static magnetic field when correction based on the angle θ is performed as in, for example, the second embodiment or the modification thereof.

### Fourth Embodiment

In the first embodiment, the anisotropy model is determined with the premise that the main axis direction of diffusion coefficient and the axis for which the characteristic value of electrical conductivity is maximized coincide to each other. However, the relation between the main axis direction of diffusion coefficient and electrical conductivity may differ depending on the presence or absence of disease, or difference of tissue or part. Therefore, this embodiment is characterized in that correlations of data of diffusion coefficient and data of electrical characteristic obtained beforehand for every part or tissue are made into a database, and the information of the database is utilized. That is, the electrical characteristic measuring apparatus of this embodiment further comprises a database that stores relations between the electrical characteristics calculated by the electrical characteristic calculation part and the continuing direction of the tissue structure used for the calculation for a plurality of tissue structures.

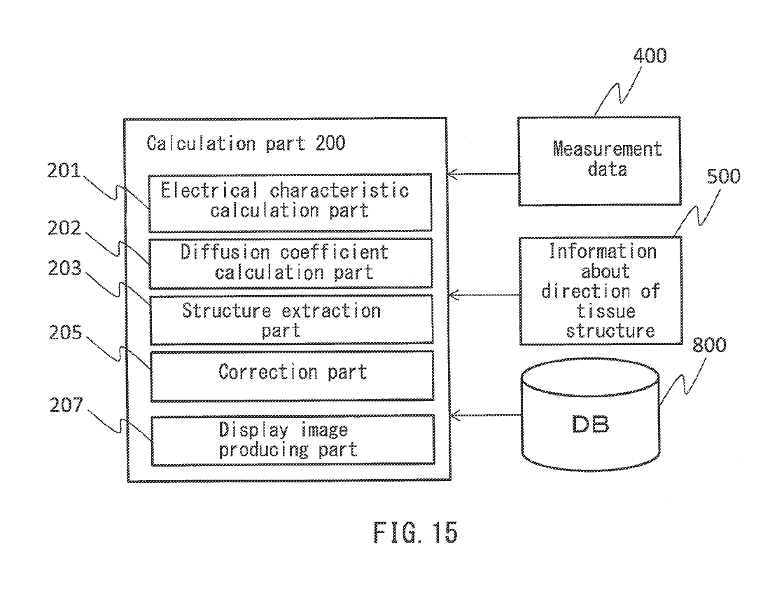

The configuration of the calculation part 200 of the electrical characteristic measuring apparatus of this embodiment is shown in FIG. 15. In FIG. 15, the same components as those of FIG. 2 are indicated with the same numerals, and detailed explanations thereof are omitted. As shown in FIG. 15, a storage device 800 that stores a database (DB) is connected to the signal processing part 120. The storage device 800 may be an external storage device, or the storage part 230, which is contained in the electrical characteristic measuring apparatus. The database stores information of electrical conductivity measured with the electrical characteristic measuring apparatus for a plurality of axes for each of a plurality of tissue structures or parts, and information of diffusion coefficient in the form of tables. These data are created by using, for example, values measured for a human phantom or actual human as the object. A normal model or disease model may further be set.

The electrical characteristic measurement of a subject as the measurement object is performed by the same procedures as those of the first embodiment mentioned above, but when the anisotropy model of electrical conductivity is determined after the measurement of diffusion coefficient (FIG. 7, S702 etc.), with reference to the database, the relation of corresponding diffusion coefficient and electrical conductivity is obtained from a table of the tissue as the measurement object. For example, if the main axis direction of diffusion coefficient and the direction of axis for which the characteristic value of electrical conductivity is maximized are the same, an anisotropy model of electrical conductivity is determined in the same manner as that of the first embodiment as shown in FIG. 8. When the main axis direction of diffusion coefficient and the direction of the axis for which the characteristic value of electrical conductivity is maximized differ, an anisotropy model is set so that the major axis direction of the anisotropy model of electrical conductivity should be the direction of the axis in which the characteristic value of electrical conductivity is maximized.

The anisotropy model to be set may be changed with reference to the database. For example, it can be judged which anisotropy model is suitable among the anisotropy models 610, 620, and 630 shown in FIG. 6 on the basis of the diffusion coefficient (tensor) of a predetermined tissue to determine the optimal anisotropy model.

It is the same as the other embodiments that after the anisotropy model is set in such a manner as described above, a subject is placed in a predetermined position, and electrical characteristic is measured. When relations between the data of electrical conductivity and the data of diffusion coefficient have been obtained for a plurality of axes as information of the database, it is also possible to perform the measurement for only one axis direction, and presume measurement results for the other axes by using the relations stored in the database. The burdens on the subject and operator for the measurement at a plurality of postures can be thereby reduced.

Although embodiments of the apparatus and method for measuring electrical characteristic of the present invention are explained above, the present invention is characterized in that the relation between anisotropy of electrical characteristic and the coordinate system of the apparatus is grasped beforehand, and electrical characteristic including anisotropy is highly precisely measured by using the relation, and is not limited to these embodiments, and various modifications are possible. For example, it is possible to omit or add a component that does not directly relate to the aforementioned characteristics of the present invention, or combine components used in the embodiments to such an extent that any technical contradiction does not occur. The functional block diagrams shown in FIG. 2 or 15 are those used for showing the functions of the signal processing part or calculation part for convenience, and do not intend to exclude a case where the functional parts are operated with one program, or one functional part is operated with a combination of a plurality of programs or hardware.

### DESCRIPTION OF NOTATIONS

110 . . . Measurement part
120 . . . Signal processing part
130 . . . Operation part
131 . . . Display part
132 . . . Input part
230 . . . Storage part
200 . . . Calculation part
201 . . . Electrical characteristic calculation part
202 . . . Diffusion coefficient calculation part
203 . . . Structure extraction part
205 . . . Correction part
207 . . . Display image producing part

What is claimed is:

1. An electrical characteristic measuring apparatus using nuclear magnetic resonance comprising:
a measurement part that measures magnetic resonance signals of a subject,
a storage part that stores information about direction of a tissue structure of the subject, and
a calculation part that calculates an electrical characteristic of a region including the tissue structure using measurement data obtained by measurement of the region performed by the measurement part, wherein:
the calculation part comprises an electrical characteristic calculation part that calculates a rotating magnetic field from the measurement data, and calculates the electrical characteristic using the rotating magnetic field,
the measurement data are measurement data measured with setting direction of the tissue structure to be a predetermined direction in a coordinate system of the apparatus, and
the electrical characteristic calculation part calculates the electrical characteristic including anisotropy using relation between the direction of the tissue structure and the predetermined direction in the coordinate system of the apparatus stored in the storage part.

2. The electrical characteristic measuring apparatus according to claim 1, wherein:
the storage part stores diffusion coefficient obtained by measurement of the region including the tissue structure performed by the measurement part as information about the direction of the tissue structure.

3. The electrical characteristic measuring apparatus according to claim 1, wherein:
the storage part stores information extracted from an image of the tissue structure as information about the direction of the tissue structure.

4. The electrical characteristic measuring apparatus according to claim 1, wherein:
the electrical characteristic calculation part determines an anisotropy model of the electrical characteristic set beforehand for the tissue structure, and calculates the electrical characteristic.

5. The electrical characteristic measuring apparatus according to claim 4, wherein:
the anisotropy model is a biaxial anisotropy model or a triaxial anisotropy model.

6. The electrical characteristic measuring apparatus according to claim 1, wherein:
the electrical characteristic calculation part calculates electrical conductivity as the electrical characteristic.

7. The electrical characteristic measuring apparatus according to claim 1, wherein:
the calculation part calculates the electrical characteristic of the region by using first measurement data obtained by the measurement part by measuring the region including the tissue structure with setting the direction of the tissue structure to be a first direction in the coordinate system of the apparatus, and second measurement data obtained by the measurement part by measuring the region including the tissue structure with setting the direction of the tissue structure to be a second direction in the coordinate system of the apparatus.

8. The electrical characteristic measuring apparatus according to claim 1, wherein:
the predetermined direction is a direction of an axis in the coordinate system for which it is desired to measure the electrical characteristic with high precision.

9. The electrical characteristic measuring apparatus according to claim 1, wherein:
the predetermined direction is a direction of a static magnetic field generated by a static magnetic field generator of the measurement part.

10. The electrical characteristic measuring apparatus according to claim 7, wherein:
the first direction is the direction of the static magnetic field generated by the static magnetic field generator of the measurement part, and the second direction is a direction crossing the direction of the static magnetic field generated by the static magnetic field generator of the measurement part.

11. The electrical characteristic measuring apparatus according to claim 1, wherein:
the calculation part further comprises a correction part that corrects the electrical characteristic calculated by using the rotating magnetic field using an angle between continuing direction of the tissue structure and the direction of the axis in the coordinate system for which the rotating magnetic field can be detected with the highest precision.

12. The electrical characteristic measuring apparatus according to claim 1, wherein:
the apparatus further comprises a database that stores relation between the electrical characteristic calculated by the electrical characteristic calculation part and continuing direction of the tissue structure used for the calculation for a plurality of tissue structures, and
the electrical characteristic calculation part determines the anisotropy model of the electrical characteristic and calculating the electrical characteristic using the relation between the electrical characteristic and the continuing direction of the tissue structure stored in the database.

13. The electrical characteristic measuring apparatus according to claim 1, wherein:
the apparatus further comprises a user interface that supports placement of a predetermined part of the subject along a predetermined direction in the coordinate system of the apparatus.

14. A method for measuring an electrical characteristic of a predetermined region of a subject placed in a static magnetic field space using magnetic resonance signals measured for the region, which comprises:
creating a rotating magnetic field map of the region from measurement data consisting of the magnetic resonance signals, and
calculating the electrical characteristic using the rotating magnetic field map, wherein the electrical characteristic is calculated as electrical characteristic including anisotropy by using measurement data measured with two or more arrangements that provide different directions of the subject with respect to the direction of the static magnetic field.

15. The method for measuring electrical characteristic according to claim 14, wherein:
when the electrical characteristic is calculated, the calculated electrical characteristic is corrected by using an angle between the direction of the static magnetic field and a predetermined direction of the tissue structure of the subject.
